(19) United States
(12) Patent Application Publication
(10) Pub. No.: US 2019/0374756 A1
(43) Pub. Date: Dec. 12, 2019

(54) Method for Treating Head or Cranium Pain Including Migraine and Cluster Headaches

(71) Applicant: James R. Winbush, Anderson, SC (US)

(72) Inventor: James R. Winbush, Anderson, SC (US)

(21) Appl. No.: 16/436,315

(22) Filed: Jun. 10, 2019

Related U.S. Application Data
(60) Provisional application No. 62/683,326, filed on Jun. 11, 2018.

(51) Int. Cl. A61M 31/00

(52) U.S. Cl. CPC A61M 31/00; A61M 2210/0618; A61M 2202/048

(57) ABSTRACT

A method for treating head or cranium pain including migraine and cluster headaches, as well as for treating acute migraine headaches, wherein a user or patient utilizes an intranasal device attached to a syringe, were with the head or cranium of the user or patient placed in a sniffing position, the product (i.e., liquid medication) is insufflated (i.e., blown) into a nasal cavity by the user or patient such that the liquid medication flows into the posterior nasopharynx and subsequently flows into the oral pharynx, and thus saturates the palatine ganglion without the use of needles, swabs or harsh medications in order to provide relief from head or cranium pain.

### CROSS-REFERENCE TO RELATED APPLICATION

This application claims priority from U.S. Provisional Application Ser. No. 62/683,326 filed Jun. 11, 2018, the content of which is incorporated herein by reference in its entirety.

### BACKGROUND OF THE INVENTION

### 1. Field of the Invention

The present invention relates to the treatment of head or cranium pain and, more particularly, to a method for treating head or cranium pain, including migraine and cluster headaches, as well as for treating acute migraine headaches.

### 2. Description of the Related Art

The Sphenopalatine Ganglion (SPG) is a group of nerve cells that is linked to the trigeminal nerve, the main nerve involved in headaches. The SPG, located behind the nose, directly under the nasal mucosa, carries information about sensation, including pain, and also plays a role in autonomic functions, such as tearing and nasal congestion.

In particular, the SPG is located in the sphenopalatine fossa, encapsulated in connective tissue, surrounded by fat tissue and separated from the nasal cavity by a bony wall. The sphenopalatine fossa communicates with the nasal cavity through the sphenopalatine foramen (SPF), which contains neurovascular structures packed with connective tissue and is covered by mucosa in the nasal cavity.

The SPF has been a target for the treatment of headache disorders for a significant number over years, in fact decades. The link between the SPG and the trigeminal nerve is important in head pain. If local anesthetics (or numbing medications) are applied to block or partially block the SPG, this can be helpful in reducing head and facial pain. Indeed, there have been attempts to block the SPG and treat a broad range of head or facial pain, such as i) cluster headaches, (ii) migraine headaches, iii) Trigeminal Neuralgia, iv) Post postherpetic trigeminal neuralgia, v) Posttraumatic headaches, vi) Post dural puncture headaches, and/or vii) Hemicrania continua.

Over a significant number of years, during which medical trials and studies were performed, a significant treatment effect in patients with migraine headaches was demonstrated by the intranasal administration of local anesthetics. In line with this hypothesis and evidence base, the application of intranasal local anesthetic as close as possible to the SPF became commercially beneficial. In the past, all procedures involving SPG and head or cranium pain were performed by physicians and medical personnel, which entailed significant costs and time expenditure.

### SUMMARY OF THE INVENTION

Disclosed is a method for treating head or cranium pain including migraine and cluster headaches, as well as for treating acute migraine headaches. In accordance with the disclosed method, a user or patient utilizes an intranasal device attached to a syringe. This device, which is sold over-the-counter (OTC) without the need for a prescription, is a modified version of a sphenopalatine ganglion block. With the head or cranium of the user or patient placed in a sniffing position, the product (i.e., liquid medication) is insufflated (i.e., blown) into a nasal cavity by the user or patient.

After the nasal cavity is bathed by the liquid medication, it flows into the posterior nasopharynx and subsequently flows into the oral pharynx, and thus saturates the palatine ganglion without the use of needles, swabs or harsh medications. The nasopharynx is an air-containing cavity that occupies the uppermost extent of the aerodigestive tract. The roof and posterior wall of the nasopharynx are formed by the sphenoid sinus, the clivus, and anterior aspect of the first two cervical vertebrae. The inferior aspect of the nasopharynx is formed by the hard palate, the soft palate, and the ridge of pharyngeal musculature that opposes the soft palate when it is elevated (Passavant's ridge). The lateral nasopharyngeal walls are formed by the margins of the superior constrictor muscle. Anteriorly, the nasopharynx is in direct continuity with the nasal cavity through the posterior choanae. The nasopharynx is in direct communication with the middle ear cavity through the eustachian tubes.

The method in accordance with the invention provides a procedure that is comfortable for users or patients, which involves a low level of risk, and is a simple OTC procedure. Advantageously, it is possible for users or patients to perform the procedure/treatment at home without the need to visit a medical facility.

After a local anesthetic is apply intra-nasally, it normally flows rapidly to the posterior nasopharynx where the anesthetic is sometimes swallowed, which does not allow enough time for absorption/diffusion through the sphenopalatine foramen into the sphenopalatine fossa to affect the SPG.

The disclosed method in accordance with the invention solves the foregoing problem by providing an increased contact time with the mucosa around the sphenopalatine foramen, which is accomplished by positioning and the holding of a breath after a deep inspiration (i.e., the drawing of air into the lungs; inhalation). The method of the invention allows closure of the upper airways which restricts fluid flow and contains the fluid in the nasal cavity. Consequently, increased contact time with the fluid and mucosa around the foramen is allowed which, in turn, allows the potential increase in diffusion and absorption.

As such, the pressure generated by the plunger of the syringe being propelled forward allows enough/increased pressure (hydrostatic) which increases the potential for fluid to diffuse through the sphenopalatine foramen.

The increased time and the increased pressure processes potentiate one another/synergistically. That is, they work better together than alone. There is also a multiplier effect with respect to anesthetic benefits. With the head or cranium in the specific sniffing position, this multiplies the already increased pressure from hydrostatic forces. Thus, with increased pressure this precludes increased diffusion/absorption and thus increased anesthesia of SPG. Increased anesthesia of the SPG means increased head pain relief.

There are multiple benefits provided by the method of the invention, e.g., (i) no needles are used, and thus less risk for trauma and/or infection, (ii) no risk factors typically associated with oral medications (renal/hepatic/addictive), (ii) safe for use during pregnancy, (iv) significant cost reduction, (v) OTC dispensing, (vi) no imaging required, and thus no radiation exposure.

Other objects and features of the present invention will become apparent from the following detailed description considered in conjunction with the accompanying drawings. It is to be understood, however, that the drawings are designed solely for purposes of illustration and not as a definition of the limits of the invention, for which reference should be made to the appended claims. It should be further understood that the drawings are not necessarily drawn to scale and that, unless otherwise indicated, they are merely intended to conceptually illustrate the structures and procedures described herein.

### BRIEF DESCRIPTION OF THE DRAWINGS

An exemplary embodiment of the invention is explained in greater detail below with reference to the drawings, these comprising schematic and greatly simplified figures in which:

FIG. 1 is an Illustration of the mucosa over the sphenopalatine foramen;

FIG. 2 is an Illustration of the relation between the nasal cavity and the sphenopalatine fossa (axial plane);

FIGS. 3a and 3b shown a syringe in position to apply the liquid medication in accordance with the method of the invention; and

FIG. 4 is a flowchart of the method in accordance with the invention.

### DETAILED DESCRIPTION OF THE EXEMPLARY EMBODIMENTS

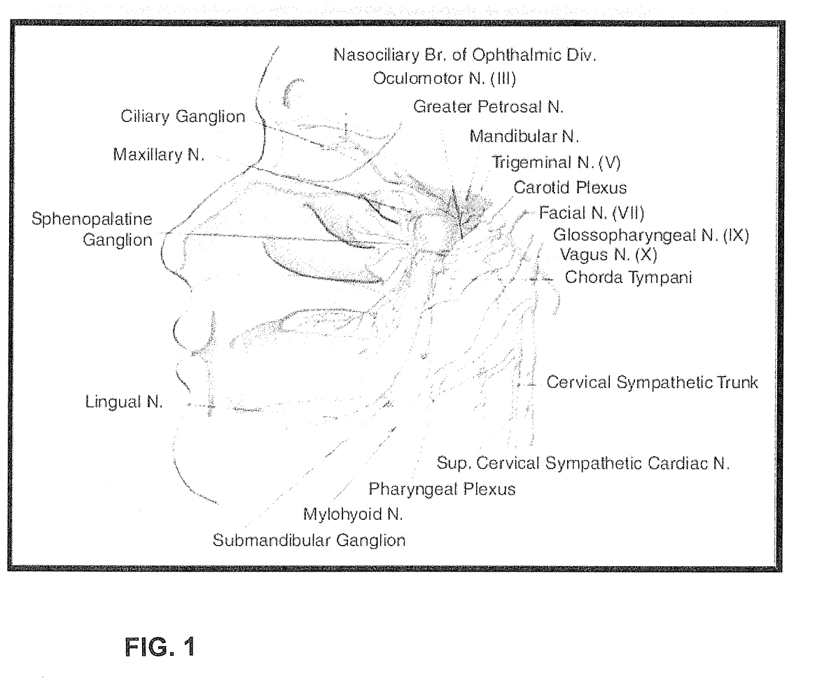

With reference to FIG. 1, the Sphenopalatine Ganglion (SPG) is a group of nerve cells that is linked to the trigeminal nerve, the main nerve involved in headaches. The SPG, located behind the nose, directly under the nasal mucosa, carries information about sensation, including pain, and also plays a role in autonomic functions, such as tearing and nasal congestion. In particular, the SPG is located in the sphenopalatine fossa, encapsulated in connective tissue, surrounded by fat tissue and separated from the nasal cavity by a bony wall. The sphenopalatine fossa communicates with the nasal cavity through the sphenopalatine foramen (SPF), which contains neurovascular structures packed with connective tissue and is covered by mucosa in the nasal cavity.

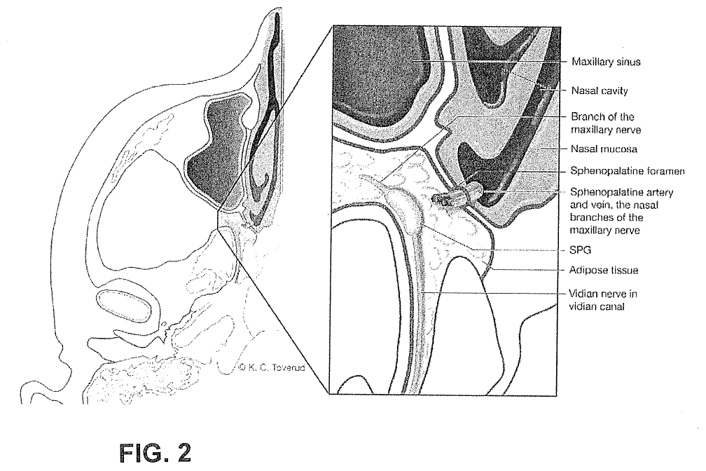

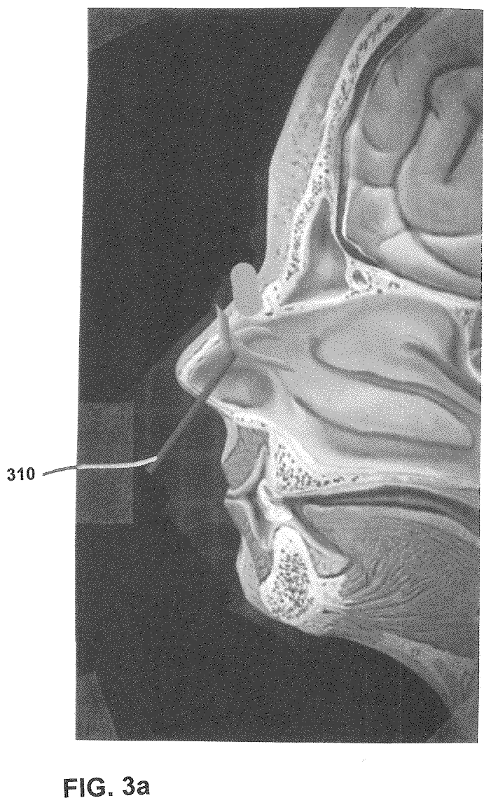

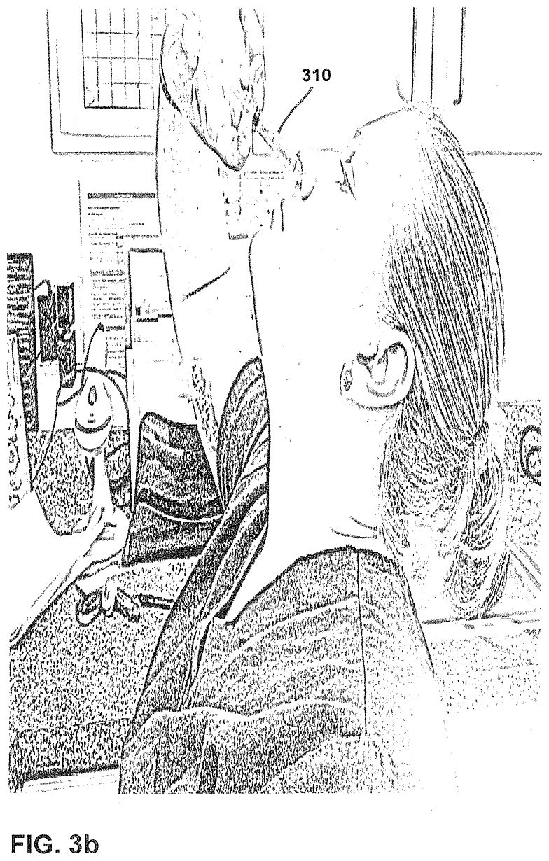

FIG. 2 is an Illustration of the relationship between a nasal cavity and the sphenopalatine fossa (axial plane). In order to reach the SPG, a drug (or medication) applied intranasally over the sphenopalatine foramen will have to diffuse through mucosa, the sphenopalatine foramen, which is packed with neuro-vascular structures and connective tissue, and the fat tissue filling the sphenopalatine fossa. In order to implement the method of the invention, the user or patient performs the following as instructed. Initially, the user or patient blows their nose to clear or eliminate the presence of nates. Next, while sitting in an upright position, the user or patient then places their head or cranium in a sniffing position (see, e.g., FIGS. 3a and 3b).

Liquid medication is now introduced into one nostril via a syringe 310, while taking a deep breath and holding this breath, which causes the closure of airways and prevents the flow of liquid (i.e., the medication) into the oral airways. After a quantity of the liquid medication is introduced into the nasal cavity via the syringe 310, the user or patient then changes their head or cranium position such that it is oriented downward, and they no longer hold their breath to allow liquid to flow into the contra lateral nostril and oral cavity. Preferably, the dosage amount is approximately 2.5 ml of the liquid medication. Secondary to holding their breath, the liquid medication stays in the nasal mucosa preventing runoff into the oral cavity and eventually into the airways.

After the user or patient ceases holding their breath, the liquid medication is allowed to flow into the oral mucosa and again into the contra lateral nostril. After this process, the liquid medication is expelled by having the user or patient blow their nose into a suitable receptacle. In order to complete the process, the procedure is repeated for the opposite nasal passage.

The method in accordance with the invention is performed without the use of imaging technology, and also with the syringe being propelled forward by the user or patient's own force. This increases hydrostatic forces, which allows increased diffusion across the mucosa, and thus assists in delivering the liquid medication to the desired area.

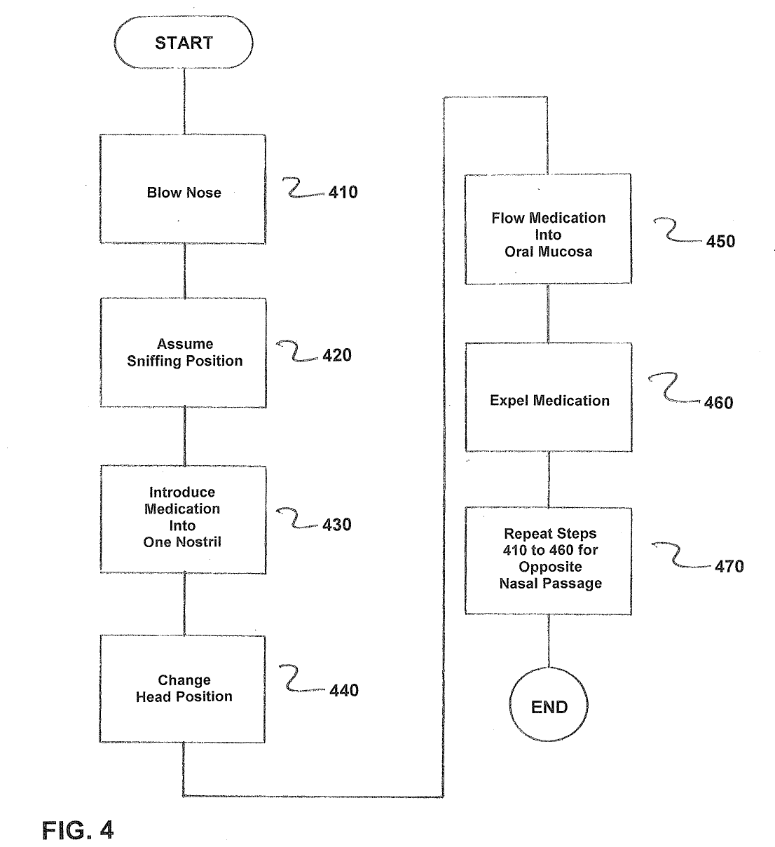

FIG. 4 is a flowchart of the method in accordance with the invention. The method comprises having a user or patient blow their nose to clear or eliminate the presence of nates, as indicated in step 410. Next, the user or patient then places their head or cranium in a sniffing position while sitting in an upright position, as indicated in step 420.

Next, liquid medication is introduced into one nostril via a syringe 310, while having the user or patient take a deep breath and holding this breath, as indicated in step 430. In accordance with the method of the invention, this causes the closure of airways and prevents the flow of liquid (i.e., the medication) into the oral airways. After a quantity of the liquid medication is introduced into the nasal cavity via the syringe 310, the user or patient then changes their head or cranium position such that it is oriented downward, and they no longer hold their breath to allow liquid to flow into the contra lateral nostril and oral cavity, as indicated in step 440. Preferably, the dosage amount of the liquid medication is approximately 2.5 ml. Secondary to holding their breath, the liquid medication stays in the nasal mucosa preventing runoff into the oral cavity and eventually into the airways.

After the user or patient ceases holding their breath, the liquid medication is allowed to flow into the oral mucosa and again into the contra lateral nostril, as indicated in step 450. The liquid medication is now expelled by having the user or patient blow their nose into a suitable receptacle, as indicated in step 460. Next, steps 410 to 460 are repeated for the opposite nasal passage of the user or patient, as indicated in step 470.

The method in accordance with the invention is performed without the use of imaging technology, and also with the syringe 310 being propelled forward by the user or patient's own force. This increases hydrostatic forces, which allows increased diffusion across the mucosa, and thus assists in delivering the liquid medication to the desired area.

In the preferred embodiment, the medication comprises 2-5% lidocaine Hcl liquid, which is the highest dose allowed for OTC and Corticosteroid (generic) comparable to 20 mg of Kenalog.

In summary, the benefits provided by the method of the invention is that it is effective for all ages and is even suitable for use during pregnancy. The method of the invention is effective for all debilitating headaches, including acute and chronic migraine headaches. Moreover, the method effectively reduces head or facial pain. It should be noted a sedating medication is not used pursuant to implementing the method of the invention. The method of the invention provides a significant decrease in patient costs in comparison to conventional procedures for treating headache or head or cranium pain. Also of note is the decreased potential side effects that are patient exposed to, as well as an absence of radiation exposure.

Thus, while there have been shown, described and pointed out fundamental novel features of the invention as applied to a preferred embodiment thereof, it will be understood that various omissions and substitutions and changes in the form and details of the methods described and the devices illustrated, and in their operation, may be made by those skilled in the art without departing from the spirit of the invention. For example, it is expressly intended that all combinations of those method steps which perform substantially the same function in substantially the same way to achieve the same results are within the scope of the invention. Moreover, it should be recognized that method steps shown and/or described in connection with any disclosed form or embodiment of the invention may be incorporated in any other disclosed or described or suggested form or embodiment as a general matter of design choice. It is the intention, therefore, to be limited only as indicated by the scope of the claims appended hereto.

What is claimed is:

1. A method for treating head or cranium pain, the method comprising:
blowing, by a user or patient, both nasal passages to clear or eliminate nates from said nasal passages;
placing a cranium of the user or patient in a sniffing position while sitting in an upright position;
introducing liquid medication into one nostril of the nasal passage via a syringe, while the user or patient takes a deep breath and holds this breath to allow closure of nasal airways and prevent flow of liquid medication into oral airways of the patient;
changing a cranium position of the user or patient such that said cranium is oriented downward, after a quantity of the liquid medication is introduced into the nasal passages via the syringe, and releasing the breath to allow liquid to flow into a contra lateral nostril and oral cavity of the patient or user;
allowing the liquid medication to flow into the oral mucosa and again into the contra lateral nostril after the user or patient ceases holding their breath; and
blowing, by the user or patient, the nasal passage to expel the liquid medication.

2. The method as claimed in step 1, wherein the method is repeated for an opposite nasal passage of the user or patient.

3. The method of claim 1, wherein a dosage amount of the liquid medication is approximately 2.5 ml.

4. The method of claim 1, wherein the liquid medication comprises 2-5% lidocaine Hcl liquid.
